(19) United States
(12) Plant Patent Application Publication
(10) Pub. No.: US 2003/0115651 P1
(43) Pub. Date: Jun. 19, 2003

(54) Sutera plant named Inuit

(75) Inventor: Hubert Brandkamp, Anholt (DE)

Correspondence Address: WOOD, PHILLIPS, KATZ, CLARK & MORTIMER, 500 W. MADISON STREET, SUITE 3800, CHICAGO, IL, 60661, US

(73) Assignee: Brandkamp GmbH

(21) Appl. No.: 10/013,786

(22) Filed: Dec. 7, 2001

(51) Int. Cl. A01H 5/00

(52) U.S. Cl. PLT/263

(57) ABSTRACT

A new and distinct Sutera plant named ‘Inuit’, characterized by its large white flowers, spreading, trailing habit, and dark green leaves.

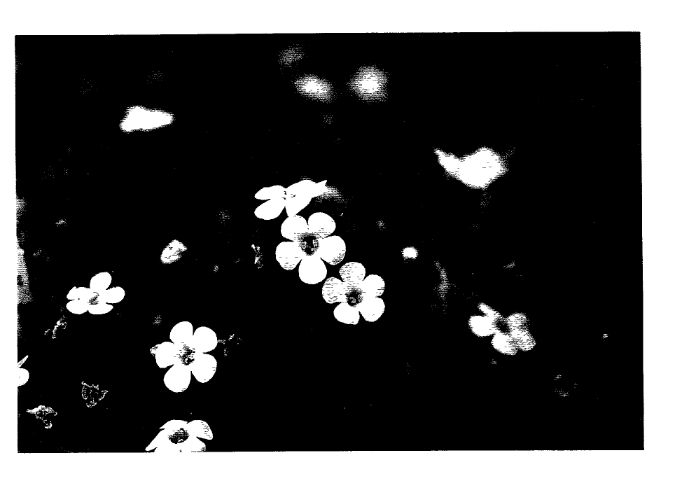

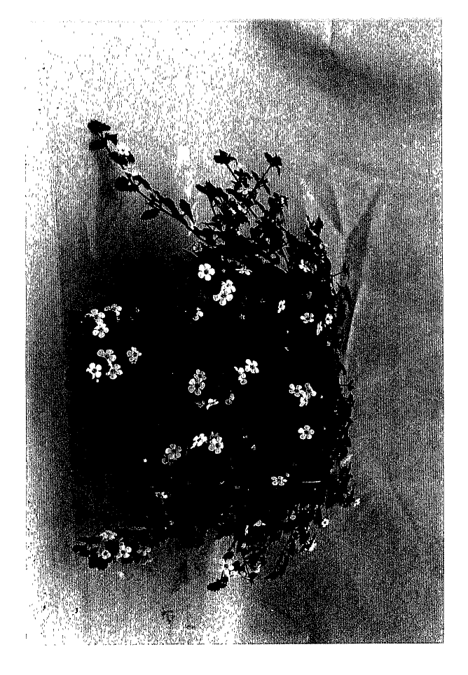

### BACKGROUND OF THE INVENTION

The present invention comprises a new and distinct Sutera plant, botanically known as Sutera cordata, and hereinafter referred to by the cultivar name Inuit.

Inuit originated as a naturally occurring mutation of the Sutera cordata cultivar Snowflake(not patented). The new cultivar was discovered by the inventor during 2000, in a controlled environment at lsselburg, Germany.

Asexual reproduction of the new cultivar by terminal tip cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits large white flowers,

(b) forms dark green foliage,

(c) exhibits a good basal branching character and

(c) exhibits a spreading and trailing growth habit.

The new cultivar of the present invention can be compared to ‘Cabana’ (not patented). In side by side comparisons, plants of the new cultivar exhibit a shorter but more spreading growth habit, have longer internodes and slightly larger leaves than ‘Cabana’, as detailed in TABLE A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown for 1 0 weeks in a greenhouse at West Chicago, Ill., U.S.A.

### DETAILED BOTANICAL DESCRIPTION

The cultivar Inuit has not been observed under all possible environmental conditions to date. Accordingly, it is possible that the phenotype may vary somewhat with variations in the environment, such as temperature, light intensity, and day length. The chart used in the identification of colors described herein is the R.H.S. Colour Chart of The Royal Horticultural Society, London, England. The color values were determined on Oct. 15, 2001. The readings were taken between 1:00 and 3:00 p.m. under natural daylight conditions. The plants were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night. Plants used for the following descriptions and measurements were grown for 10 weeks from rooted cuttings.

Classification:

Botanical.—Sutera cordata cultivar Inuit.

Parentage: Naturally occurring mutation of the Sutera cordata cultivar Snowflake (not patented).

Propagation:

Type cutting.—Terminal tip.

Time to initiate roots.—Approximately 7 to 10 days.

Time to develop roots.—Approximately 21 to 30 days.

Root description.—Fibrous, branching.

Plant Description:

Habit of growth.—Moderately vigorous with good basal branching. Pinching improves basal branching. A mature plant, 8 to 10 weeks after the planting of a rooted cutting, commonly measures approximately 7.5 cm in height and approximately 38.7 cm in diameter with an average of 4 main branches and 13.7 lateral branches.

Form.—Spreading and trailing.

Stem.—Strong and woody. Approximately 1 mm in diameter, densely pubescent and 144A. Internode length at middle of branch is approximately 2.8 cm.

Foliage.—Leaves are non-fragrant, single, opposite, at an acute angle to the stem, and broadly elliptic with serrate margin, acute apex and attenuate base. Upper and lower surfaces are sparsely pubescent with lower veins moderately pubescent. Leaf length is approximately 1.9 cm and width is approximately 2.5 cm. Upper surface of both young and mature foliage is 137A, lower surface of both young and mature foliage is 137C. Both upper and lower surfaces have pinnate venation closest to 144A. Petiole length is approximately 9 mm, diameter is approximately 2 mm, surface is densely pubescent and color is closest to 143C.

Flowering Description:

Flowering habit.—Freely flowering.

Natural flowering season.—Year round in greenhouse environment; April through October in outdoor garden.

Flower type.—Solitary, axillary, slaverform, sympetalous, actinomorphic. Flowers are persistent.

Peduncle.—Strong, densely pubescent with acute angle, approximately 6 mm in length, less than 1 mm in diameter. Peduncle color is 144A.

Bud.—Tubular, approximately 6 mm in length, 3 mm in diameter. Bud, just before opening, is 155D at distil end with small area of 23B at center of corolla tube and 145D at base of tube.

Flower description.—Non-fragrant. Corolla is round, approximately 1.5 cm in diameter composed of five (5) flat lobes. Lobes have rounded tips, are approximately 6 mm long and 6 mm wide and have an entire margin. Color of upper surface of lobes when flower is opening and when fully opened is 155DD. Color of lower surface of lobes both when opening and when fully opened is 155D. Corolla tube is approximately 4 mm in diameter, 9 mm in length and pubescent on both inner and outer surface. Color of inner surface: distil ¼, is 144D, center ½ is 14A and last ¼, at opening, is 155D. Color of outer surface: distil ¼, is 144D, center ½ is 21C and last ¼, at opening, is 155D.

Calyx.—Five parted tube fused at base. Approximately 8 mm in length. Sepals are linear, less than 1 mm in width, have an acute apex, entire margin. The both surfaces are densely pubescent and 143A.

Reproductive organs.—Androecium: There are 4 stamens — 2 are 8 mm in length, 2 are 6 mm in length. Anthers are fan shaped and 1 mm in length. Pollen amount is moderate and color is 13A. Gynoecium: One pistil, 1.1 cm in length. Stigma shape is spatulate, 2 mm in length and 144C. Style length is 8 mm, color is 1D. Ovary diameter is 1 mm, color is 143A.

Seed production: Seed production has not been observed.

Disease resistance: Resistance to pathogens is not claimed

TABLE A

| CHARACTERISTIC | INUIT | CABANA |
|---|---|---|
| Mature leaf length × | 1.9 cm × 2.5 cm | 1.4 cm × 2 cm |
| width | | |
| Internode length | 2.8 cm | 2 cm |
| Plant height | 7.5 cm | 11 cm |
| Plant diameter | 38.7 cm | 30 cm |

I claim:

1. A new and distinct cultivar of Sutera cordata named ‘Inuit’ substantially as herein shown and described, which:
(a) exhibits large white flowers;
(b) dark green foliage;
(c) exhibits a good basal branching character; and
(d) a spreading and trailing growth habit.
